(19) United States
(12) Patent Application Publication
(10) Pub. No.: US 2011/0263907 A1
(43) Pub. Date: Oct. 27, 2011

(54) PROCESS FOR PREPARING 1,6-HEXANEDIOL

(75) Inventors: Olivier Abillard, Mannheim (DE); Gerd-Dieter Tebben, Mannheim (DE); Rolf Pinkos, Bad Duerkheim (DE); Tobias Wabnitz, Mannheim (DE)

(73) Assignee: BASF SE, Ludwigshafen (DE)

(21) Appl. No.: 13/133,006

(22) PCT Filed: Nov. 30, 2009
(86) PCT No.: PCT/EP09/66011

§ 371 Date: Jun. 6, 2011

(30) Foreign Application Priority Data
Dec. 5, 2008 (EP) 08170853.9

(51) Int. Cl. C07C 31/20

(52) U.S. Cl. 568/852

(57) ABSTRACT

A process for preparing 1,6-hexanediol from a carboxylic acid mixture which comprises adipic acid, 6-hydroxycaproic acid and small amounts of 1,4-cyclohexanediols and is obtained as a by-product of the oxidation of cyclohexane to cyclohexanone/cyclohexanol with oxygen or oxygen-comprising gases by water extraction of the reaction mixture, by esterification and hydrogenation to hexanediol, which comprises
a) the mono- and dicarboxylic acids present in the aqueous reaction mixture are reacted with a low molecular weight alcohol to give the corresponding carboxylic esters,
b) the resulting esterification mixture is freed of excess alcohol and low boilers in a first distillation stage,
c) in a second distillation stage, a separation is performed of the bottom product into an ester fraction essentially free of 1,4-cyclohexanediols and a fraction comprising at least the majority of the 1,4-cyclohexanediols,
d) the ester fraction from (c) is catalytically hydrogenated and 1,6-hexanediol is obtained in a manner known per se by distilling the hydrogenation product,
e) optionally the bottom product of stage c) is subjected to a further esterification reaction with a low molecular weight alcohol, the low molecular weight alcohol is distillatively removed on completion of the esterification reaction, then adipic diesters and 6-hydroxycaproic esters are substantially separated from 1,4-cyclohexanediols and high boilers in the remaining bottom stream by distillation and said adipic diesters and 6-hydroxycaproic esters are fed to stage c),
   which comprises performing at least one of the esterification reactions with a catalyst which comprises at least one element of groups 3-14, and adding a monomeric or oligomeric polyol with at least 3 hydroxyl functions after the esterification reaction.

After step c) of the process according to the invention, in a third distillation stage, a stream comprising essentially methyl 6-hydroxycaproate can be at least partly removed and heated to temperatures above 200° C. under reduced pressure, which cyclizes 6-hydroxycaproic ester to caprolactone, and pure ε-caprolactone can be obtained from the cyclization product by distillation.

The invention relates to a process for preparing 1,6-hexanediol, preferably with at least 99% purity, which is especially virtually free of 1,4-cyclohexanediols, from a carboxylic acid mixture which is obtained as a by-product of the oxidation of cyclohexane to cyclohexanone/cyclohexanol with oxygen or oxygen-comprising gases and by water extraction of the reaction mixture, by esterification and hydrogenation to hexanediol, wherein the yield of products of value is enhanced by, after an esterification stage in which catalysts which comprise at least one element of groups 3-14 are used, adding a monomeric or polymeric polyol with at least 3-hydroxyl functions.

1,6-hexanediol is a sought-after monomer unit which is used predominantly in the polyester and polyurethane sector.

The aqueous solutions of carboxylic acids, which arise as by-products in the oxidation of cyclohexane to cyclohexanol and cyclohexanone (cf. Ullmann's Encyclopedia of Industrial Chemistry, 5th ed., 1987, vol. A8, p. 49), referred to hereinafter as dicarboxylic acid solution (DCS), comprise (calculated without water in % by weight) generally between 10 and 40% adipic acid, between 10 and 40% 6-hydroxycaproic acid, between 1 and 10% glutaric acid, between 1 and 10% 5-hydroxyvaleric acid, between 1 and 5% 1,2-cyclohexanediols, between 1 and 5% 1,4-cyclohexanediols, between 2 and 10% formic acid and a multitude of further mono- and dicarboxylic acids, esters, oxo and oxa compounds, the individual contents of which generally do not exceed 5%. Examples include acetic acid, propionic acid, butyric acid, valeric acid, caproic acid, oxalic acid, malonic acid, succinic acid, 4-hydroxybutyric acid and γ-butyrolactone.

DE 2 321 101 and DE 1 235 879 disclose hydrogenating these aqueous dicarboxylic acid solutions at temperatures of 120 to 300° C. and pressures of 50 to 700 bar in the presence of catalysts comprising predominantly cobalt to give 1,6-hexanediol as the main product. The hydrogenation discharges are preferably worked up by distillation. Even with an extremely high level of distillation complexity, it is possible to remove the 1,4-cyclohexanediols unchanged in the hydrogenation only incompletely, if at all, from 1,6-hexanediol, such that the 1,4-cyclohexanediols which were already present initially in the DCS are found again with a content of generally 2 to 5% by weight in the 1,6-hexanediol.

In order to counter this problem, some approaches to a solution are known:

U.S. Pat. No. 3,933,930 describes the conversion of 1,4-cyclohexanediol in aqueous solutions of adipic acid and 6-hydroxycaproic acid to cyclohexanol, cyclohexane and/or cyclohexene, by catalytically prehydrogenating the mixture. This process requires the use of two different hydrogenation catalysts, one for the prehydrogenation, one for the actual carboxylic acid hydrogenation, and is therefore costly and inconvenient.

According to DE-A 2 060 548 very pure 1,6-hexanediol is obtained by crystallization. This process too is very complex and is also associated with considerable yield losses.

A further means of obtaining high-purity 1,6-hexanediol consists in hydrogenating, instead of DCS, pure adipic acid or pure adipic esters (K. Weissermel, H. J. Arpe, Industrielle Organische Chemie [Industrial Organic Chemistry], VCH-Verlagsgemeinschaft Weinheim, 4th edition, page 263, 1994). However, pure adipic acid is very expensive compared to DCS. In addition, the carboxylic acid mixture obtained in the cyclohexane oxidation is a waste product which should be sent to a material utilization for environmental reasons among others.

EP-B 883591 describes an elegant process which ensures the preparation of 1,6-hexanediol and caprolactone in high purities. A disadvantage of this process is the non-optimal exploitation of the adipic acid and 6-hydroxycaproic acid components present in the carboxylic acid mixture (DCS), which was obtained by cyclohexane oxidation, since some of them do not leave the process as 1,6-hexanediol and caprolactone respectively, but as high-boiling oligomeric esters.

It was therefore the object of the present invention to obtain very pure 1,6-hexanediol from the adipic acid present in the DCS, and to increase the yield of 1,6-hexanediol and hence the disadvantages of the prior art, i.e. either high costs of preparation or insufficient purity of the products.

This object was achieved by a process for preparing 1,6-hexanediol from a carboxylic acid mixture which comprises adipic acid, 6-hydroxycaproic acid and small amounts of 1,4-cyclohexanediols and is obtained as a by-product of the oxidation of cyclohexane to cyclohexanone/cyclohexanol with oxygen or oxygen-comprising gases and by water extraction of the reaction mixture, by esterification and hydrogenation to hexanediol, which comprise
a) the mono- and dicarboxylic acids present in the aqueous reaction mixture are reacted with a low molecular weight alcohol to give the corresponding carboxylic esters,
b) the resulting esterification mixture is freed of excess alcohol and low boilers in a first distillation stage,
c) in a second distillation stage, a separation is performed of the bottom product into an ester fraction essentially free of 1,4-cyclohexanediols and a fraction comprising at least the majority of the 1,4-cyclohexanediols,
d) the ester fraction from (c) is catalytically hydrogenated and 1,6-hexanediol is obtained in a manner known per se by distilling the hydrogenation product,
e) optionally the bottom product of stage c) is subjected to a further esterification reaction with a low molecular weight alcohol, the low molecular weight alcohol is distillatively removed on completion of the esterification reaction, then adipic diesters and 6-hydroxycaproic esters are substantially separated from 1,4-cyclohexanediols and high boilers in the remaining bottom stream by distillation and said adipic diesters and 6-hydroxycaproic esters are fed to stage c),
   which comprises performing at least one of the esterification reactions with a catalyst which comprises at least one element of groups 3-14, and adding a monomeric or oligomeric polyol with at least 3 hydroxyl functions after the esterification reaction.

After step c) of the process according to the invention, in a third distillation stage, a stream comprising essentially methyl 6-hydroxycaproate can be at least partly removed and heated to temperatures above 200° C. under reduced pressure, which cyclizes 6-hydroxycaproic ester to caprolactone, and pure c-caprolactone can be obtained from the cyclization product by distillation.

The expression in step c) of the process according to the invention that, in a second distillation stage, there is a separation into an ester fraction essentially free of 1,4-cyclohexanediols means that this top fraction comprises less than 0.5% by weight of 1,4-CHDO, preferably less than 0.2% by weight of 1,4-CHDO, more preferably less than 0.1% by weight of 1,4-CHDO. The expression in step c) of the process according to the invention that, in a second distillation stage, the removal is effected into a fraction comprising at least the majority of the 1,4-cyclohexanediols means that this fraction comprises more than 75% by weight of the fractions of 1,4-cyclohexanediols present in the bottom product before the second distillation stage.

It was surprising that, in the course of separation of the ester mixtures which arise through the esterification of the mono- and dicarboxylic acids present in the DCS, the 1,4-cyclohexanediols, which may likewise be present esterified with carboxylic acids, can be removed in such a way that, after hydrogenation and workup, the very small 1,4-cyclohexanediol content remaining in the 1,6-hexanediol is virtually no longer of any practical significance. Owing to the complicated substance mixtures for separation, it was surprising that it was possible to remove the 1,4-cyclohexanediols or esters thereof, in spite of the unfavorable boiling point ratios and risk of azeotrope formation, virtually fully from the C6 esters used for the hydrogenation to 1,6-hexanediol. Furthermore, it was surprising that addition of a monomeric or oligomeric polyol with at least 3 hydroxyl functions, after at least one esterification stage, allowed the yield of C6 products of value such as 1,6-hexanediol and/or caprolactone to be enhanced. It was likewise surprising that no decomposition products of the polyol formed, which can lead to problems in further process steps, whether owing to accumulations or owing to components which have toxic action or because they constitute new secondary components in 1,6-hexanediol and/or caprolactone.

At least one esterification of the process according to the invention is performed in the presence of a catalyst which comprises a transition metal; otherwise, the esterification can be performed without addition of catalysts, preferably with the action of catalysts. Useful low molecular weight alcohols generally include those having 1 to 10 carbon atoms, preferably alcohols having 1 to 8 carbon atoms, more preferably alcohols having 1 to 3 carbon atoms. Diols such as butanediol or pentanediol are also useful in principle.

The industrially preferred alcohols for use for the esterification are methanol, n- or i-butanol and most preferably methanol.

In the case of esterification with methanol, the procedure is to obtain, in the distillation stage (c), a methyl carboxylate fraction essentially free of 1,4-cyclohexanediols at the top of the column and a bottom fraction comprising the high boilers and the 1,4-cyclohexanediols, and to continue to use the methyl carboxylate fraction in step d). In addition, it is possible to vary the process according to the invention in such a way that a portion of the methyl carboxylate fraction is also utilized to prepare ε-caprolactone.

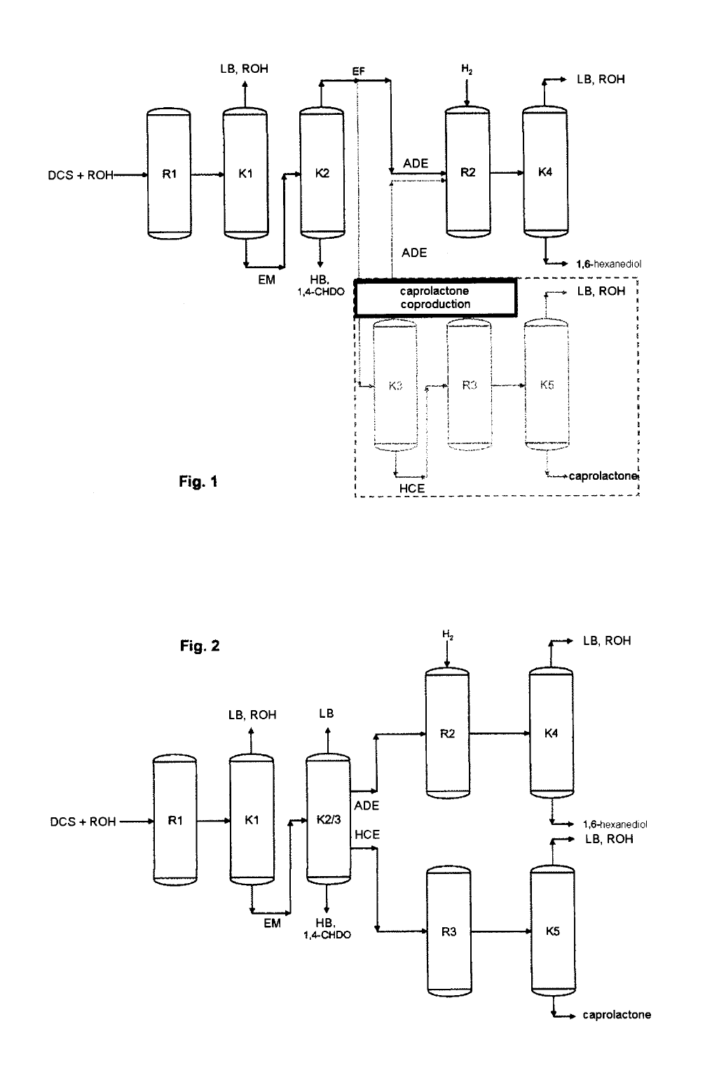

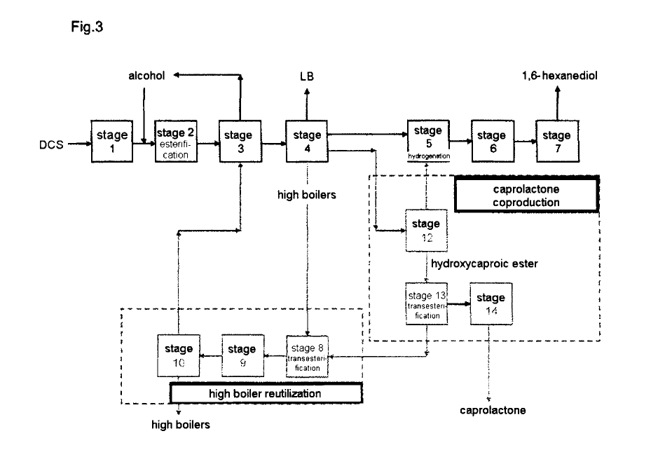

In the case of esterification with a particularly preferred low molecular weight alcohol with 1 to 3 carbon atoms, the process according to the invention is explained as follows in general terms as variant A (the terms “via the top” and “as bottoms” in each case meaning draw removal above and below the feed respectively). FIGS. 1 and 3 reproduce the process according to the invention according to variant A, also depicting the further utilization of the fraction which comprises essentially 1,4 cyclohexanediol and arises from step c) according to step e) and the distillative removal of a stream comprising essentially 6-hydroxycaproic ester which follows step c), and the further workup thereof to caprolactone. In the case of further processing of the stream comprising essentially 6-hydroxycaproic ester to caprolactone, the process according to the invention can be conducted according to variant B. FIG. 2 specifies the process according to the invention according to variant B.

### VARIANT A

As shown in FIG. 1, the dicarboxylic acid solution (DCS), optionally after dewatering, is fed together with a C₁- to C₃-alcohol, preferably methanol, into the esterification reactor R₁ in which the carboxylic acids are esterified. The resulting esterification mixture then passes into the column K₁, in which the excess alcohol (ROH), water and low boilers (LB) are distilled off via the top and the ester mixture (EM) is drawn off as bottoms and fed into the column K₂. In this column, the EM is fractionated into an ester fraction (EF) essentially free of 1,4-cyclohexanediols and a bottom fraction consisting of high boilers (HB) and cis- and trans-1,4-cyclohexanediols (1,4-CHDO), which is optionally worked up further in step f) of the process according to the invention. The ester fraction is subsequently hydrogenated in the catalytic hydrogenation R2 to 1,6-hexanediol, which is subjected to purifying distillation in column K4.

ε-caprolactone can additionally also be obtained from the process according to the invention. For this purpose, the process according to variant A is modified as follows: the ester fraction from column K2 is then passed into a further fractionating column K₃ in which the ester fraction is separated into a top product consisting essentially of adipic diester (ADE), preferably dimethyl adipate; and a bottom product consisting essentially of 6-hydroxycaproic ester (HCE), preferably methyl 6-hydroxycaproate. The 6-hydroxycaproic ester fraction from the bottom product of the fractionating column K₃ can be subjected in the reactor R₃ to a thermal treatment above 100° C., generally 150 to 350° C., preferably 200 to 300° C., under reduced pressure, for example, 900 to 10 mbar, preferably 300 to 20 mbar; this leads to cyclization of the ester to form ε-caprolactone, which can be subjected to purifying distillation in column K₅.

### VARIANT B

In this variant, the distillation of columns K₂ and K₃ is combined to give one distillation stage.

According to FIG. 2, the ester mixture (EM) obtained by esterification with alcohols having 1 to 3 carbon atoms, preferably methanol, is subjected to a fractional distillation and the adipic ester, preferably dimethyl adipate, is obtained in an upper side draw, the 6-hydroxycaproic ester, preferably methyl 6-hydroxycaproate, in a lower side draw, and the 1,4-cyclohexanediols and other oligomeric high boilers as bottoms. The bottoms fraction is worked up further in stage f).

The adipic ester and 6-hydroxycaproic ester fractions are then worked up as described in FIG. 1.

The process according to the invention is illustrated hereinafter specifically for variant A with reference to FIG. 3. The reaction conditions apply equally to the other variant.

The process steps are broken down into stages in FIG. 3, stages 2, 3, 4, 5, 6, 7 being essential to the process, stages 8, 9, 10 being optional for enhancing the yield—where necessary—and stages 3 and 4 and 6 and 7 also being combinable. Stage 11 is optional but may be advisable to increase the economic viability of the process. If caprolactone is also to be prepared with the 6-hydroxycaproic ester fraction obtained during the process according to the invention, steps 12-14 are also essential.

The dicarboxylic acid solution (DCS) is generally an aqueous solution with a water content of 20 to 80%. Since an esterification reaction is an equilibrium reaction in which water forms, it is advisable, especially in the case of esterification with methanol for example, to remove water present before the reaction, in particular when water cannot be removed, for example cannot be removed azeotropically, during the esterification reaction. The dewatering in stage 1 can be effected, for example, with a membrane system, or preferably by means of a distillation apparatus, in which, at 10 to 250° C., preferably 20 to 200° C., particularly 30 to 200° C. and a pressure of 1 to 1500 mbar, preferably 5 to 1100 mbar, more preferably 20 to 1000 mbar, water is removed via the top, and higher monocarboxylic acids, dicarboxylic acids and 1,4-cyclohexanediols via the bottom. The bottom temperature is preferably selected such that the bottom product can be drawn off in liquid form. The water content in the bottom of the column may be 0.01 to 10% by weight, preferably 0.01 to 5% by weight, more preferably 0.01 to 1% by weight.

The water can be removed in such a way that the water is obtained in predominantly acid-free form, or the lower monocarboxylic acids—essentially formic acid—present in the DCS can for the most part be distilled off with the water, in order that they do not bind any esterification alcohol in the esterification.

Alcohol ROH with 1 to 10 carbon atoms is added to the carboxylic acid stream from stage 1. The alcohol used may be methanol, ethanol, propanol or isopropanol or mixtures of the alcohols, but preferably methanol. The mixing ratio of alcohol to carboxylic acid stream (mass ratio) can be from 0.1 to 50, preferably 0.2 to 40, more preferably 0.5 to 30.

This mixture passes as a melt or solution into the reactor of stage 2, in which the carboxylic acids are esterified with the alcohol. The esterification reaction can be performed at 50 to 400° C., preferably 70 to 300° C., more preferably 90 to 200° C. An external pressure can be applied, but the esterification is preferably performed under the autogenous pressure of the reaction system. The esterification apparatus used may be a stirred tank or flow tube, or it is possible to use a plurality of each. Likewise possible is a reaction column in which dicarboxylic acid solution and alcohol are reacted with one another in countercurrent such that the DCS is introduced in the upper part of the reaction column and descends; alcohol is usually introduced in the gaseous state from the bottom and ascends. Excess alcohol is drawn off via the top together with water of reaction, and the esterified carboxylic acids via the bottom. The residence time needed for the esterification is between 0.3 and 10 hours, preferably 0.5 to 5 hours. The esterification reaction can proceed without addition of a catalyst, but preference is given to adding a catalyst to increase the reaction rate. This may be a homogeneous dissolved catalyst or a solid catalyst. Examples of homogeneous catalysts include sulfuric acid, phosphoric acid, hydrochloric acid, sulfonic acids such as p-toluenesulfonic acid, heteropolyacids such as tungstophosphoric acid, or Lewis acids, for example, aluminum compounds, vanadium compounds, titanium compounds, boron compounds. Preference is given to mineral acids, especially sulfuric acid. When a reaction column is used, a preferred variant is to use catalysts which comprise elements such as Ti, Zr, V, Hf, Al, Sn. It is additionally preferred to ensure a portion of the reaction autocatalytically, and the remainder catalytically. The weight ratio of homogeneous catalyst to carboxylic acid melt is generally 0.0001 to 0.5, preferably 0.001 to 0.3.

Suitable solid catalysts are acidic or superacidic materials, for example acidic and superacidic metal oxides such as SiO₂, Al₂O₃, SnO₂, ZrO₂, sheet silicates or zeolites, all of which may be doped with mineral acid residues such as sulfate or phosphate for acid strengthening, or organic ion exchangers with sulfonic acid or carboxylic acid groups. The solid catalysts may be arranged as a fixed bed or used as a suspension.

The water formed in the reaction is appropriately removed continuously, for example by means of a membrane or by distillation.

The completeness of the conversion of the free carboxyl groups present in the carboxylic acid melt is determined with the acid number (mg KOH/g) measured after the reaction. Minus any acid added as a catalyst, it is 0.01 to 50, preferably 0.1 to 10. Not all carboxyl groups present in the system need be present as esters of the alcohol used, but a portion may be present in the form of dimeric or oligomeric esters, for example with the OH-end of the hydroxycaproic acid.

The esterification mixture is fed into stage 3, preferably a distillation column. If a dissolved acid was used as the catalyst for the esterification reaction, the esterification mixture is appropriately neutralized with a base, 1 to 1.5 base equivalents being added per acid equivalent of the catalyst. The bases used are generally alkali metal or alkaline earth metal oxides, carbonates, hydroxides or alkoxides, or amines in bulk or dissolved in the esterification alcohol.

When the acid number is below 1 mg KOH/g, preferably below 0.2, and very preferably below 0.1, in the case of esterification in the process according to the invention by means of a catalyst comprising at least one element of groups 3-14, a monomeric or oligomeric polyol with at least 3 hydroxyl functions should be added before the entry of the product stream into a column of stage 3, but no later than stage 4. The monomeric or oligomeric polyols are selected from the group of glycerol, pentaerythritol, 1,1,1-trimethylolpropane, erythritol, pentoses, hexoses, sorbitol, lower or higher starches and cellulose, particular preference being given to glycerol. These monomeric or oligomeric polyols are used in amounts of 0.01 to 20% by weight, preferably 0.05 to 5% by weight, more preferably 0.1 to 3% by weight, based on the stream to be distilled. They can also be used in the form of mixtures, in pure form or else in dissolved form. Suitable solvents are, for example, water, the esterification alcohol, preferably methanol, and additionally glycols or glycol ethers or tetrahydrofuran.

When a column is used in stage 3, the feed to the column is preferably between the top stream and the bottom stream. The excess esterification alcohols ROH, water and corresponding esters of formic acid, acetic acid and propionic acid are drawn off via the top at pressures of 1 to 1500 mbar, preferably 20 to 1000 mbar, more preferably 40 to 800 mbar, and temperatures between 0 and 150° C., preferably 15 and 90° C. and especially 25 and 75° C. This stream can either be combusted or preferably subjected to further workup in stage 11.

The bottoms obtained are an ester mixture which consists predominantly of the esters of the alcohol ROH used with dicarboxylic acids such as adipic acid and glutaric acid, hydroxycarboxylic acids such as 6-hydroxycaproic acid and 5-hydroxyvaleric acid, and of oligomers and free or esterified 1,4-cyclohexanediols. It may be advisable to permit a residual content of water and/or alcohol ROH up to 4% by weight in each case in the ester mixture. The bottom temperatures are 70 to 250° C., preferably 80 to 220° C., more preferably 100 to 190° C.

The stream from stage 3 which has been substantially freed of water and esterification alcohol ROH is fed into stage 4. This is a distillation column, in which the feed is between the low-boiling components and the high-boiling components. The column is operated at temperatures of 10 to 300° C., preferably 20 to 270° C., more preferably 30 to 250° C. and pressures of 1 to 1000 mbar, preferably 5 to 500 mbar, more preferably 10 to 200 mbar.

The top fraction consists predominantly of residual water and residual alcohol ROH, esters of the alcohol ROH with monocarboxylic acids, predominantly C3- to C6-monocarboxylic esters with hydroxycarboxylic acids, such as 6-hydroxycaproic acid, 5-hydroxyvaleric acid, and in particular the diesters with dicarboxylic acids such as adipic acid, glutaric acid and succinic acid, 1,2-cyclohexanediols, caprolactone and valerolactone. This top fraction which is essentially free of 1,4-cyclohexanediols comprises less than 0.5% by weight of 1,4-CHDO, preferably less than 0.2% by weight of 1,4-CHDO, more preferably less than 0.1% by weight of 1,4-CHDO.

The components mentioned can be removed together via the top or, in a further preferred embodiment, separated in the column of stage 4 into a top stream which comprises predominantly residual water and residual alcohol, and the abovementioned constituents having 3 to 5 carbon atoms, and a side stream which comprises predominantly the abovementioned constituents of the C₆ esters. The stream comprising the esters of the C₆ acids, either as an overall top stream or as a side stream, can then, according to how much caprolactone is to be prepared, pass entirely into the hydrogenation (stage 5) in the process according to the invention—without caprolactone preparation—but optionally also be fed partly or as the entire stream into stage 12.

The high-boiling components of the stream from stage 4, predominantly consisting of 1,4-cyclohexanediols or esters thereof, dimeric or oligomeric esters and constituents of the DCS which are not defined in detail, some of them being polymeric, are removed via the stripping section of the column of stage 4. These may be obtained together or in such a way that predominantly the 1,4-cyclohexanediols are removed via a side stream of the column in the stripping section, and the remainder via the bottom. The 1,4-cyclohexanediols thus obtained may find use, for example, as a starting material for active ingredients.

When the yield of desired C6 esters after the process according to the invention is already high enough, stages 8, 9 and 10 can be dispensed with. Should, though, the preceding stages described not be configured in accordance with the invention, i.e. the esterification is not performed with a catalyst which comprises at least one element of groups 3-14, followed by the addition of a polyol, stages 8 to 10 are then necessary.

To this end, in the process according to the invention, the bottom product of stage 4 is subjected to a further esterification reaction. Since quite predominantly oligomeric esters are converted to monomeric esters by means of an alcohol and of a catalyst in this stage 8, this stage is also referred to as “transesterification stage”.

To this end, in stage 8, the proportion of dimeric and oligomeric esters of adipic acid or hydroxycaproic acid is reacted with further amounts of the alcohol ROH, preferably methanol, in the presence of a catalyst which comprises at least one element of groups 3-14. The weight ratio of alcohol ROH and the bottom stream from stage 4 is between 0.1 and 20, preferably 0.5 to 10, more preferably 1 to 5. Suitable catalysts are compounds or complexes, for example, of aluminum, of tin, of antimony, of zirconium or of titanium, such as zirconium acetylacetonate or tetraalkyl titanate such as tetraisopropyl titanate, which are employed in concentrations of 1 to 10 000 ppm, preferably 50 to 6000 ppm, more preferably 100 to 4000 ppm. Particular preference is given to titanium compounds.

The transesterification can be performed batchwise or continuously, in one reactor or a plurality of reactors, series-connected stirred tanks or tubular reactors, or a reaction column, at temperatures between 100 and 300° C., preferably 120 to 270° C., more preferably 140 to 240° C., and the autogenous pressures established. The residence times required are 0.5 to 10 hours, preferably 1 to 4 hours.

The reaction effluent of stage 8 is freed of excess ROH in a subsequent distillation (stage 9), the methanol being removed via the top in the case that ROH=methanol. According to the invention, before the entry of the product stream into a column of stage 9, but no later than stage 10, a monomeric or oligomeric polyol with at least 3 hydroxyl functions is added. The monomeric or oligomeric polyols are selected from the group of glycerol, pentaerythritol, 1,1,1-trimethylolpropane, erythritol, pentoses, hexoses, sorbitol, lower or higher starches and cellulose, particular preference being given to glycerol. These monomeric or oligomeric polyols are used in amounts of 0.01 to 20% by weight, preferably 0.05 to 5% by weight, more preferably 0.1 to 3% by weight, based on the stream to be distilled. They can also be used in the form of mixtures in pure form or else in dissolved form. Suitable solvents are, for example, water, the esterification alcohol, preferably methanol, and additionally glycols or glycol ethers or tetrahydrofuran.

In stage 9, the column is operated such that the feed to the column is preferably between the top stream and the bottom stream.

The excess esterification alcohol is drawn off via the top at pressures of 1 to 1500 mbar, preferably 20 to 1000 mbar, more preferably 40 to 800 mbar, and temperatures between 0 and 150° C., preferably 15 and 90° C. and especially 25 and 75° C., and recycled, for example, into stage 11 or into stage 2 or stage 8.

The bottom stream of stage 9 is transferred into stage 10, likewise a distillation column. This is a distillation column in which the feed is between the low-boiling components and the high-boiling components. The column is operated at temperatures of 10 to 300° C., preferably 20 to 270° C., more preferably 30 to 250° C. and pressures of 1 to 1000 mbar, preferably 5 to 500 mbar, more preferably 10 to 200 mbar. Essentially a mixture of adipic diester and 6-hydroxycaproic ester is obtained via the top; the bottoms comprise predominantly 1,4-cyclohexanediols and for the most part unknown high boilers, and the polyol added. The top product of stage 10 can be fed either to stage 4 or to stage 12.

With the inventive method, it is possible to obtain the adipic acid and 6-hydroxycaproic acid units present in the DCS in higher yields, and hence to enhance the yields of 1,6-hexanediol and caprolactone. The process according to the invention has a yield-enhancing effect especially on the 6-hydroxycaproic ester. For instance, the yield of the monomeric C6 esters can be enhanced, for example, by 5 to 25%.

Stages 3 and 4 can be combined, especially when only relatively small amounts are processed. To this end, the C₆ ester stream can be obtained, for example, in a batchwise fractional distillation again without 1,4-cyclohexanediols getting into the stream conducted to the hydrogenation.

When only 1,6-hexanediol is to be obtained after the process, the top product or that from the side draw of stage 4, if appropriate together with the top product of stage 10, can be converted directly without further purification, in a hydrogenation.

The hydrogenation is effected catalytically, either in the gas phase or liquid phase. Useful catalysts in principle include all homogeneous and heterogeneous catalysts suitable for hydrogenation of carbonyl groups, such as metals, metal oxides, metal compounds or mixtures thereof. Examples of homogeneous catalysts are described in H. Kropf, Houben-Weyl, Methoden der Organischen Chemie [Methods of Organic Chemistry], Volume IV/1c, Georg Thieme Verlag Stuttgart, 1980, p. 45 to 67, and examples of heterogeneous catalysts are described in Houben-Weyl, Methoden der Organischen Chemie, Volume IV/1c, p. 16 to 26.

Preference is given to using catalysts which comprise one or more of the elements from transition groups I and VI to VIII of the Periodic Table of the Elements, preferably copper, chromium, molybdenum, manganese, rhenium, ruthenium, cobalt, nickel and palladium, more preferably copper, cobalt or rhenium.

The catalysts may consist solely of the active components or the active components may be applied to supports. Suitable support materials are, for example, Cr₂O₃, Al₂O₃, SiO₂, ZrO₂, TiO₂, ZnO₂, BaO and MgO or mixtures thereof.

Preference is given to using heterogeneous catalysts which are either used in fixed bed form or as a suspension. When the hydrogenation is performed in the gas phase and over fixed bed catalyst, generally temperatures of 150 to 300° C. are employed at pressures of 1 to 50 bar. The amount of hydrogen used as a hydrogenating agent and carrier gas is at least sufficient that reactants, intermediates and products never become liquid during the reaction.

When the hydrogenation is effected in the liquid phase with fixed bed or suspended catalyst, it is generally performed at temperatures between 100 and 350° C., preferably 120 and 300° C. and pressures of 30 to 350 bar, preferably 40 to 300 bar.

The hydrogenation can be performed in one reactor or a plurality of reactors connected in series. The hydrogenation in the liquid phase over a fixed bed can be performed either in trickle mode or liquid phase mode. In a preferred embodiment, a plurality of reactors are used, the predominant portion of the esters being hydrogenated in the first reactor and the first reactor being operated preferably with liquid circulation for heat removal, and the downstream reactor (reactors) preferably being operated without circulation to complete the conversion.

The hydrogenation can be effected batchwise, preferably continuously.

The hydrogenation discharge consists essentially of 1,6-hexanediol and the alcohol ROH. Further constituents are in particular, if the entire low-boiling stream of stage 4 has been used, 1,5-pentanediol, 1,4-butanediol, 1,2-cyclohexanediols and small amounts of monoalcohols having 1 to 6 carbon atoms and water.

The hydrogenation discharge is separated in stage 6, for example a membrane system or preferably a distillation column, into the alcohol ROH, which additionally comprises the majority of the further low-boiling components, and a stream which comprises predominantly 1,6-hexanediol as well as 1,5-pentanediol and the 1,2-cyclohexanediols. In this separation, at a pressure of 10 to 1500 mbar, preferably 30 to 1200 mbar, more preferably 50 to 1000 mbar, top temperatures of 0 to 120° C., preferably 20 to 100° C., more preferably 30 to 90° C. and bottom temperatures of 100 to 270° C., preferably 140 to 260° C., more preferably 160 to 250° C., are established. The low-boiling stream can either be recycled directly into the esterification of stage 2 or pass into stage 8 or into stage 11.

The stream comprising 1,6-hexanediol is purified in stage 7 in a column. In this purification, 1,5-pentanediol, the 1,2-cyclohexanediols and any further low boilers present are removed via the top. If the 1,2-cyclohexanediols and/or 1,5-pentanediol are to be obtained as additional products of value, they can be separated in a further column. Any high boilers present are discharged via the bottom. 1,6-Hexanediol is withdrawn from a side stream of the column with a purity of at least 99%. In this column, at pressures of 1 to 1000 bar, preferably 5 to 800 mbar, more preferably 20 to 500 mbar, top temperatures of 50 to 200° C., preferably 60 to 150° C., and bottom temperatures of 130 to 270° C., preferably 150 to 250° C., are established.

If only smaller amounts of 1,6-hexanediol are to be prepared, stages 6 and 7 can also be combined in a batchwise fractional distillation.

In order to operate the process according to the invention in a very economically viable manner, it is advisable to recover the esterification alcohol ROH and to always use it again for esterification. To this end, the stream comprising predominantly the alcohol ROH from stage 3 and/or 6 can be worked up in stage 11. To this end, it is advantageous to use a column in which components which have lower boiling points than the alcohol ROH are removed via the top, water and components which have higher boiling points than the alcohol ROH via the bottom, from the alcohol ROH, which is obtained in a side stream. The column is operated appropriately at 500 to 5000 mbar, preferably at 800 to 3000 mbar.

A preferred process variant envisages, in addition to the preparation of 1,6-hexanediol, also the recovery of caprolactone. To this end, for the caprolactone preparation, the stream comprising predominantly esters of the C₆ acids from stage 4 is used. To this end, this stream is separated in stage 12, a distillation column, into a stream which comprises predominantly adipic diesters and comprises the 1,2-cyclohexanediols present via the top, and a stream which comprises predominantly 6-hydroxycaproic esters and does not comprise any 1,2-cyclohexanediols via the bottom. The column is operated at pressures of 1 to 500 mbar, preferably 5 to 350 mbar, more preferably 10 to 200 mbar and bottom temperatures of 80 to 250° C., preferably 100 to 200° C., more preferably 110 to 180° C. The top temperatures are established correspondingly.

What is important for a high purity and high yield of caprolactone is the removal of the 1,2-cyclohexanediols from the hydroxycaproic ester, since these components form azeotropes with one another. It was not foreseeable in this stage 12 that the separation of the 1,2-cyclohexanediols and of the hydroxycaproic ester succeeds completely, in particular when the ester used is the preferred methyl ester.

The bottom stream comprising 6-hydroxycaproic esters from stage 12 is converted in stage 13 in a manner known per se, either in the gas phase or liquid phase, to alcohol and caprolactone. Preference is given to the liquid phase.

The reaction is performed without catalyst or else preferably in the presence of a catalyst. Suitable catalysts are acidic or basic catalysts which may be present in homogeneously dissolved or heterogeneous form. Examples are alkali metal and alkaline earth metal hydroxides, oxides, carbonates, alkoxides or carboxylates, acids such as sulfuric or phosphoric acid, organic acids such as sulfonic acids, or mono- or dicarboxylic acids or salts of the aforementioned acids, Lewis acids, preferably from main groups III and IV and transition groups Ito VIII of the Periodic Table of the Elements.

Preference is given to using the same catalysts which are also used in stage 8, since the high-boiling discharge stream of stage 13 comprises oligomeric hydroxycaproic acid units, which can advantageously be reutilized via stage 8. When a heterogeneous catalyst is used, the catalyst hourly space velocity is typically 0.05 to 5 kg of reactant/l of catalyst per hour. In the case of homogeneous catalysts, the catalyst is preferably added to the reactant stream. The concentration is typically 10 to 10 000 ppm, preferably 50 to 5000 ppm, more preferably 100 to 1000 ppm. The reaction is performed typically at 150 to 400° C., preferably 180 to 350° C., more preferably 190 to 330° C. and pressures of 1 to 1020 mbar, preferably 5 to 500 mbar, more preferably 10 to 200 mbar.

In some cases, it is advantageous to perform the cyclization reaction in the presence of high-boiling mono-, di- or polyols, for example decanol, undecanol, 1,4-butanediol, 1,5-pentanediol, 1,6-hexanediol, 1,4-cyclohexanediols or glycerol.

These high-boiling alcohols or polyols are initially charged or added to the reaction mixture, in each case in concentrations of 1 to 20 000 ppm, preferably 10 to 4000 ppm, more preferably 50 to 2000 ppm.

When the cyclization is performed in the liquid phase, the reaction products, predominantly esterification alcohol ROH and caprolactone, are removed from the reaction mixture in gaseous form. A column attached to the reaction vessel is advantageous, in which as yet unconverted reactant can be kept within the reaction system and the alcohol and caprolactone can be drawn off via the top. The product stream can be condensed in the manner of fractional condensation, i.e. first predominantly caprolactone, then the esterification alcohol. Of course, it is also possible to obtain only the alcohol via the top, but caprolactone in a side stream. The alcohol stream can advantageously be recycled into stage 2, 8 or 11. The bottom product of the cyclization can be introduced into stage 8.

The caprolactone product stream of stage 13 is worked up further in stage 14. This may comprise one or more columns. When one column is used, any esterification alcohol still present and other C₁ to C₆ low boilers are removed via the top, pure caprolactone via a side stream, and any unconverted hydroxycaproic ester, which is recycled, via the bottom.

High-purity caprolactone is obtained when, in stage 14, the low boilers mentioned are fed into a first column via the top, and caprolactone and other high boilers into a second column via the bottom, where caprolactone is drawn off via the top. When the caprolactone stream to be obtained is only in relatively small amounts, caprolactone can be obtained with one column by batchwise fractional distillation.

The distillations are performed at bottom temperatures of 70 to 250° C., preferably 90 to 230° C., more preferably 100 to 210° C., and pressures of 1 to 500 mbar, preferably 5 to 200 mbar, more preferably 10 to 150 mbar.

In the process according to the invention, yields of 1,6-hexanediol and caprolactone of in each case more than 95% are achieved, at purities of more than 99%.

The process is illustrated in detail by the examples which follow but in no way restricted thereby. The examples of series 1 are based on esterification in a flow tube with sulfuric acid as a catalyst, and the series is divided into inventive and noninventive. The examples of series 2 are based on an esterification in a reaction column.

### EXAMPLES SERIES 1

### Example a (Comparative Example)

### Stage 1: (Dewatering)

0.1 kg/h of dicarboxylic acid solution (adipic acid, 6-hydroxycaproic acid, 1,4-cyclohexanediols, glutaric acid, 5-hydroxyvaleric acid, formic acid, water) was distilled continuously in a distillation apparatus (three-tray bubble-cap tray column with external oil heating circuit, oil temperature 150° C., tray volume in each case approx. 25 ml, feed via the bubble-cap trays) with an attached column with random packing (approx. 4 theoretical plates, no reflux at the top). The top product obtained was 0.045 kg/h with a formic acid content in the water of approx. 3%. In the bottom stream (total of 6 kg) the water content was approx. 0.4%.

### Stage 2: (Esterification)

5.5 kg of the bottom stream from stage 1 were reacted with 8.3 kg of methanol and 14 g of sulfuric acid at approx. 120° C. in a flow tube with a residence time of approx. 3 hours. The acid number of the discharge minus sulfuric acid was approx. 10 mg KOH/g.

### Stage 3:

In a column, the esterification stream from stage 2, from which the sulfuric acid had been removed, was distilled (1015 mbar, top temperature 65° C., up to bottom temperature 125° C.). 7.0 kg were drawn off via the top. The bottom product obtained was 6.8 kg.

### Stage 4: (1,4-Cyclohexanediol Removal)

In a 50 cm column with random packing, the bottom stream from stage 3 was fractionally distilled (1 mbar, top temperature 70-90° C., up to bottom temperature 180° C.). In the bottoms, as well as unknown high boilers, were dimeric and oligomeric esters based on adipic acid and 6-hydroxycaproic acid, and the 1,4-cyclohexanediols.

As low boilers, 0.6 kg was distilled off (1,2-cyclohexanediols, valerolactone, methyl 5-hydroxyvalerate, dimethyl glutarate, dimethyl succinate, inter alia); as the fraction comprising predominantly dimethyl adipate and methyl 6-hydroxycaproate, 4.3 kg were obtained.

### Stage 5: (Substream Hydrogenation)

2.7 kg of C₆ ester mixture from stage 4 were hydrogenated continuously in a 25 ml reactor over a catalyst (catalyst: 70% by weight of CuO, 25% by weight of ZnO, 5% by weight of Al₂O₃, which has been activated beforehand in a hydrogen stream at 180° C., hydrogenation conditions: feed 20 g/h, no circulation, 220 bar, 220° C.). The ester conversion was 99.5%, the 1,6-hexanediol selectivity was more than 99%.

### Stages 6 and 7: (Hexanediol Purification)

2.5 kg of the hydrogenation discharge from stage 5 were fractionally distilled (distillation still with attached 70 cm column with random packing, reflux ratio 2). At 1013 mbar, 0.5 kg of methanol was distilled off and, after applying reduced pressure (20 mbar); predominantly the 1,2-cyclohexanediols and 1,5-pentanediol distilled off. Thereafter, 1,6-hexanediol (b.p. 146° C.) distilled off with a purity of 99.8%.

### Stage 8:

2.9 kg of the bottom discharge from stage 4 were admixed with 3.8 kg of methanol and 3.8 g of tetra-i-propyl titanate, and converted continuously in a tubular reactor of length 1 m and capacity 440 ml, which had been filled with 3 mm V2A rings. The mean residence time was approx. 2 h.

### Stage 9:

The discharge from stage 8 was fractionally distilled analogously to the apparatus described in stage 3. At top temperature 65° C., 3.5 kg were distilled off (predominantly methanol). 2.2 kg remained in the bottom.

Stage 10:

The bottom stream from stage 9 was continuously distilled in a column. The feed (80 g/h) was supplied above the bottom. At the top, the distillate obtained was partly recycled (reflux ratio 1.3:1). The bottom level was kept constant by means of a valve and continuous bottoms discharge. The distillation apparatus was operated under the following conditions: pressure 18 mbar, bottom temperature 180° C., top temperature 110° C.

The distillate obtained (35 g/h) consisted principally of methyl 6-hydroxycaproate (48% by weight) and dimethyl adipate (29% by weight). The high boilers, among other substances, consisted predominantly of unknown components, 1,4-cyclohexanediols and oligomeric esters based on 6-hydroxycaproic acid and adipic acid. The yield of methyl 6-hydroxycaproate from stage 10 was 65%, and that of dimethyl adipate 99%, based on the proportion of monomeric ester in the bottoms of stage 9.

### Stage 11:

7 kg of the top product of stage 3 were fractionally distilled at 1015 mbar on a 20 cm column with random packing. 0.8 kg of first runnings fraction was obtained at top temperature 59-65° C. and comprised, in addition to predominantly methanol, C₁-C₄-monoethyl esters. At top temperature 65° C., 5.6 kg of methanol were obtained with a purity of >99%. The bottoms (0.6 kg) consisted predominantly of water.

### Stage 12:

In a 4 l distillation still with attached column (40 cm, 5 mm V2A metal ring random packings) and reflux divider, predominantly dimethyl adipate and methyl 6-hydroxycaproate were distilled off at 2 mbar from 2.0 kg of ester mixture from stage 4 and the top product of stage 10, (reflux ratio 2, top temperature up to 91° C., bottom temperature up to 118° C.). In the bottom remained 0.5 kg of methyl hydroxycaproate (85%, remainder predominantly dimeric methyl hydroxycaproate, no dimethyl adipate).

### Stage 13: (Cyclization)

A 250 ml distillation still with external heating and attached column (70 cm, 5 mm V2A metal ring random packings) with reflux divider was initially charged with 60 ml of bottom product from stage 12 with addition of 1000 ppm of tetraisopropyl titanate and heated to 260° C. at 40 mbar, and 35 ml of bottom product from stage 12, to which 1000 ppm of tetraisopropyl titanate and 200 ppm of 1,6-hexanediol had been added, were fed in hourly. At a top temperature of 123 to 124° C. and a reflux ratio of 4, predominantly caprolactone was condensed at 25° C., and methanol at −78° C.

### Stage 14: (Caprolactone Purification)

In a 250 ml distillation still with attached column (70 cm, 5 mm V2A metal ring random packings) and reflux divider (reflux ratio 4), the caprolactone obtained from stage 13 was fractionally distilled at 40 mbar. After removal of essentially valerolactone (b.p. 90 to 110° C.), caprolactone (b.p. 131° C.) was obtained in a purity (GC area%) of 99.9%.

### EXAMPLES SERIES 1

### Example b (Inventive Example)

The method of stages 1 to 14 from the comparative example of series 1 was repeated, with the difference that 1% by weight of glycerol had been added before stage 10. At pressure 18 mbar and a bottom temperature of 180° C., a top temperature of 115° C. was established. The distillate obtained consisted principally of methyl 6-hydroxycaproate (57% by weight) and dimethyl adipate (25% by weight). The yield of methyl 6-hydroxycaproate was 90%, and that of dimethyl adipate 99%.

The top product was processed further analogously to stages 12, 13 and 14 of the comparative example. There was no change in the yield or composition of the caprolactone.

### EXAMPLES SERIES 2

### Example c (Comparative Example)

### Stage 1: (Dewatering)

0.1 kg/h dicarboxylic acid solution (adipic acid, 6-hydroxycaproic acid, 1,4-cyclohexanediols, glutaric acid, 5-hydroxyvaleric acid, formic acid, water) was distilled continuously in a distillation apparatus (three-tray bubble-cap tray column with external oil heating circuit, oil temperature 150° C., tray volume in each case approx. 25 ml, feed via the bubble-cap trays) with an attached column with random packing (approx. 4 theoretical plates, no reflux at the top). The top product obtained was 0.045 kg/h with a formic acid content in the water of approx. 3%. In the bottom stream (total of 6 kg) the water content was approx. 0.4%.

### Stage 2: (Esterification)

A total of 6 kg of the bottom stream from stage 1 was esterified continuously with methanol in countercurrent in a 10-tray bubble-cap tray column with 10 different external heating circuits at temperatures between 180 and 160° C. The acid mixture was pumped to the second highest bubble-cap tray, and methanol was pumped to the lowermost bubble-cap tray and had been heated beforehand to 180° C. Based on the acid feed, 2000 ppm of tetra-i-propyl titanate dissolved in a 10% solution in methanol were pumped to the 5 bubble-cap trays. At a reflux ratio of 5, predominantly methanol, water and low boilers such as methyl formate were removed via the top; an ester mixture which had an acid number of approximately 0.2 mg KOH/g and, as well as the esters, also comprised approx. 5% methanol was obtained via the bottom.

### Stage 3:

In a column, 5 kg of the ester product from stage 2 were distilled (1015 mbar, top temperature 65-70° C., up to bottom temperature 125° C.). Approx. 0.5 kg was drawn off via the top, predominantly methanol. The bottom product obtained was approx. 4.5 kg.

### Stage 4: (1,4-Cyclohexanediol Removal)

The bottom stream from stage 3 was continuously distilled in a column. The feed was fed in above the bottom. At the top, the distillate obtained was partly recycled (reflux ratio approx. 1). The bottom level was kept constant by means of a valve and continuous bottoms discharge. The distillation apparatus was operated under the following conditions: pressure 20 mbar, bottom temperature up to 170° C., top temperature up to 105° C. In the top product were 1,2-cyclohexanediols, valerolactone, methyl 5-hydroxyvalerate, dimethyl glutarate, dimethyl succinate, caprolactone and dimethyl adipate (approx. 50% yield based on the dimethyl adipate present in the feed) and methyl 6-hydroxycaproate (approx. 25% based on the methyl 6-hydroxycaproate present in the feed). Approx. 1.5 kg of distillate were obtained. The bottom product consisted of 1,4-cyclohexanediols, unknown high boilers and dimeric or oligomeric esters of adipic acid and 6-hydroxycaproic acid. A total of approx. 2 kg of bottom product was obtained. The remainder was composed of holdup in the column and uncondensed methanol.

The top product was hydrogenated and worked up analogously to stages 5, 6 and 7. 1,6-Hexanediol was obtained in a purity up to 99.8% in a yield at this purity, based on the methyl 6-hydroxycaproate and dimethyl adipate present after stage 2, of approx. 20%.

### EXAMPLES SERIES 2

### Example c (Inventive Example)

### Stage 1: (Dewatering)

0.1 kg/h of dicarboxylic acid solution (adipic acid, 6-hydroxycaproic acid, 1,4-cyclohexanediols, glutaric acid, 5-hydroxyvaleric acid, formic acid, water) was distilled continuously in a distillation apparatus (three-tray bubble-cap tray column with external oil heating circuit, oil temperature 150° C., tray volume approx. 25 ml each, feed via the bubble-cap trays) with an attached column with random packing (approx. 4 theoretical plates, no reflux at the top). The top product obtained was 0.045 kg/h with a formic acid content in the water of approx. 3%. In the bottom stream (total of 6 kg) the water content was approx. 0.4%.

### Stage 2: (Esterification)

A total of 6 kg of the bottom stream from stage 1 was esterified continuously with methanol in countercurrent in a 10-tray bubble-cap tray column with 10 different external heating circuits at temperatures between 180 and 160° C. The acid mixture was pumped to the second highest bubble-cap tray, and methanol was pumped to the lowermost bubble-cap tray and had been heated beforehand to 180° C. Based on the acid feed, 2000 ppm of tetra-i-propyl titanate dissolved in a 10% solution in methanol were pumped to the 5 bubble-cap trays. At a reflux ratio of 5, predominantly methanol, water and low boilers such as methyl formate were removed via the top; an ester mixture which had an acid number of approximately 0.2 mg KOH/g and, as well as the esters, also comprised approx. 5% methanol was obtained via the bottom.

### Stage 3:

2% by weight of glycerol were added to the discharge from stage 2 and then 5.1 kg were distilled in a column (1015 mbar, top temperature 65-69° C., up to bottom temperature 125° C.). Approx. 0.3 kg was drawn off via the top, predominantly methanol. The bottom product obtained was approx. 4.9 kg.

### Stage 4: (1,4-Cyclohexanediol Removal)

The bottom stream from stage 3 was continuously distilled in a column. The feed was fed in above the bottom. At the top, the distillate obtained was partly recycled (reflux ratio approx. 1). The bottoms level was kept constant by means of a valve and continuous bottoms discharge. The distillation apparatus was operated under the following conditions: pressure 20 mbar, bottom temperature up to 170° C., top temperature up to 115° C. In the top product were 1,2-cyclohexanediols, valerolactone, methyl 5-hydroxyvalerate, dimethyl glutarate, dimethyl succinate, caprolactone and dimethyl adipate (approx. 90% yield based on the dimethyl adipate present in the feed) and methyl 6-hydroxycaproate (approx. 80% based on the methyl 6-hydroxycaproate present in the feed). Approx. 3.0 kg of distillate were obtained. The bottom product consisted of the 1,4-cyclohexanediols, unknown high boilers and dimeric or oligomeric esters of adipic acid and 6-hydroxycaproic acid. A total of approx. 0.9 kg of bottom product was obtained. The remainder was composed of holdup in the column and uncondensed methanol.

The top product was hydrogenated and worked up analogously to stages 5, 6 and 7. 1,6-Hexanediol was obtained in a purity up to 99.8% in a yield at this purity, based on the methyl 6-hydroxycaproate and dimethyl adipate present after stage 2, of approx. 80%.

1. A process for preparing 1,6-hexanediol from a carboxylic acid mixture which comprises adipic acid, 6-hydroxycaproic acid and small amounts of 1,4-cyclohexanediol and is obtained as a by-product of the oxidation of cyclohexane to cyclohexanone/cyclohexanol with oxygen or oxygen-comprising gases by water extraction of the reaction mixture, by esterification and hydrogenation to hexanediol, which comprises
a) reacting the mono- and dicarboxylic acids present in the aqueous reaction mixture with an alcohol having 1 to 10 carbon atoms to give the corresponding carboxylic esters,
b) freeing the resulting esterification mixture of excess alcohol and low boilers in a first distillation stage,
c) in a second distillation stage, performing a separation of the bottom product into an ester fraction comprising less than 0.5% by weight of 1,4-cyclohexanediols and a fraction comprising at least the majority of the 1,4-cyclohexanediols,
d) catalytically hydrogenating the ester fraction from (c), from which 6-hydroxycaproic ester has been at least partly removed, and obtaining 1,6-hexanediol in a manner known per se by distilling the hydrogenation product,
e) optionally subjecting the bottom product of stage c) to a further esterification reaction with an alcohol having 1 to 10 carbon atoms, removing this alcohol by distillation on completion of the esterification reaction, then substantially separating adipic diesters and 6-hydroxycaproic esters from 1,4-cyclohexanediols and high boilers in the remaining bottom stream by distillation and feeding said adipic diesters and 6-hydroxycaproic esters to stage c),
which comprises performing at least one of the esterification reactions with a catalyst which comprises at least one element of groups 3-14, and adding glycerol after the esterification reaction.

2. The process according to claim 1, wherein the carboxylic acid mixture is dewatered before the esterification.

3. The process according to claim 1, wherein the esterification is performed with alcohols having 1 to 3 carbon atoms.

4. The process according to claim 1, wherein the esterification is performed with alcohols having 4 to 10 carbon atoms.

5. The process according to claim 1, wherein the esterification is performed with methanol and, in distillation stage (c), a methyl carboxylate fraction comprising less than 0.5% by weight of 1,4-cyclohexanediols is obtained at the top of the column and a bottom fraction is obtained comprising the high boilers and the 1,4-cyclohexanediols.

6. The process according to claim 1, wherein the esterification is performed with n- or i-butanol and, in distillation stage (c), the 1,4-cyclohexanediols are removed via the top with the low boilers and the butyl carboxylates are obtained as a sidestream or as bottoms comprising them.

7. The process according to claim 5, wherein, in the case of esterification with methanol, a fraction comprising essentially methyl dicarboxylates is removed in an upper side draw, an essentially methyl 6-hydroxycaproate fraction as a lower side draw and a fraction comprising the 1,4-cyclohexanediols as a bottom product.

8. The process according to claim 6, wherein, in the case of esterification with n- or i-butanol, a fraction comprising essentially butyl 6-hydroxycaproate is obtained in an upper side draw, a fraction comprising essentially butyl dicarboxylates as a lower side draw, and a fraction comprising the 1,4-cyclohexanediols as a top product.

9. The process according to claim 1, wherein the catalyst in esterification reaction a) is a titanium compound.

10. The process according to claim 1, wherein the catalyst in esterification reaction e) is a titanium compound in a concentration of 1 to 10,000 ppm.
